(19) United States
(12) Patent Application Publication
(10) Pub. No.: US 2016/0239621 A1
(43) Pub. Date: Aug. 18, 2016

(54) SYSTEM AND METHOD ENABLING THE EFFICIENT MANAGEMENT OF TREATMENT PLANS AND THEIR REVISIONS AND UPDATES

(71) Applicant: KONINKLIJKE PHILIPS N.V., Eindhoven (NL)

(72) Inventors: ANCA IOANA DANIELA BUCUR, EINDHOVEN (NL); RICHARD VDOVJAK, EINDHOVEN (NL)

(21) Appl. No.: 15/028,086

(22) PCT Filed: Oct. 16, 2014
(86) PCT No.: PCT/IB2014/065350

§ 371 Date: Apr. 8, 2016

Related U.S. Application Data
(60) Provisional application No. 61/894,451, filed on Oct. 23, 2013.

(51) Int. Cl. G06F 19/00; A61B 5/00

(52) U.S. Cl. CPC G06F 19/345; G06F 19/3418; A61B 5/4848

(57) ABSTRACT

A system and method for managing an executable medical treatment plan. The system and method perform the steps of analyzing one of treatment patterns and prior treatment patterns in view of patient information to generate treatment plan recommendations using a processor, receiving a user input regarding the treatment plan recommendations via a user interface and generating a current defined treatment plan based on the treatment plan recommendations and the user input regarding the treatment plan recommendations using the processor.

### BACKGROUND

Traditionally, treatment plans have been written documents that outline the progression of therapy for a given patient. Treatment plans can be described at different levels of detail using different representations that range from a text document, a table, a graph or other graphical representation, or a combination of representations. Treatments plans are introduced in order to standardize and improve the predictability and the outcome of the prescribed treatment and, by doing so, improve the standard of care and avoid errors. For example, treatment plans communicate the purpose of a given treatment to all parties involved in the process including patients, relatives, referral sources and accrediting bodies. They provide a measure and timeline for a patient's progress in treatment and keeps the entire treatment team informed of the status of the treatment. Thus, problems identified at assessment are not forgotten and appropriate follow up measures can be taken. Treatment plans also allow changes and deviations to be identified and recorded so that patients are always informed with respect to their treatment steps, changes and progression. Treatment plans may also alert the clinician when treatment is ineffective and should be modified.

To obtain these desired benefits, treatment plans need to be accurate and updated whenever changes are required. A cumbersome and time consuming process, however, make it difficult for the clinicians to keep up with changes in the treatment plan so that the clinicians may skip recording changes and/or record changes with insufficient detail. Such a cumbersome process also raises costs and increases the risk of errors. Another problem is the current lack of integrated feedback concerning executed treatment plans. Improved feedback of executed plans may help predict the need for a revision and identify relevant extensions which can be used for the elaboration of more detailed treatment plans. Additionally, by using portions of previously executed treatment plans, the elaboration of new plans may be more efficient.

### SUMMARY OF THE INVENTION

A method for managing an executable medical treatment plan. The method including analyzing one of treatment patterns and prior treatment patterns in view of patient information to generate treatment plan recommendations using a processor, receiving a user input regarding the treatment plan recommendations via a user interface and generating a current defined treatment plan based on the treatment plan recommendations and the user input regarding the treatment plan recommendations using the processor.

A system for managing an executable medical treatment plan. The system including a memory storing treatment patterns in a treatment patterns database and a set of instructions and a processor executing the instructions which causes the processor to perform operations including analyzing one of treatment patterns stored in the treatment patterns database and prior treatment patterns in view of patient information to generate treatment plan recommendations using a processor, receiving a user input regarding the treatment plan recommendations and generating a current defined treatment plan based on the treatment plan recommendations and the user input regarding the treatment plan recommendations using the processor.

### BRIEF DESCRIPTION OF THE DRAWINGS

FIG. 1 shows a schematic drawing of a system according to an exemplary embodiment;

FIG. 2 shows another schematic drawing of the system of FIG. 1.

FIG. 3 shows a flow diagram of a method according to an exemplary embodiment.

FIG. 4 shows a schematic drawing of an exemplary treatment plan according to the method of the exemplary embodiment.

FIG. 5 shows a schematic drawing of an exemplary updated treatment plan according to the method of the exemplary embodiment.

### DETAILED DESCRIPTION

The exemplary embodiments may be further understood with reference to the following description and the appended drawings wherein like elements are referred to with the same reference numerals. The exemplary embodiments relate to a system and method for managing and updating treatment plans. In particular, the exemplary embodiments describe generating a machine-executable treatment plan based on previously implemented treatment plans and the evolving needs of the patient. Although the exemplary embodiments describe treatment plans for oncology patients, it will be understood by those of skill in the art that the system and method of the present disclosure may be used to generate treatment plans for patients having any of a variety of disease or conditions within any of a variety of hospital departments.

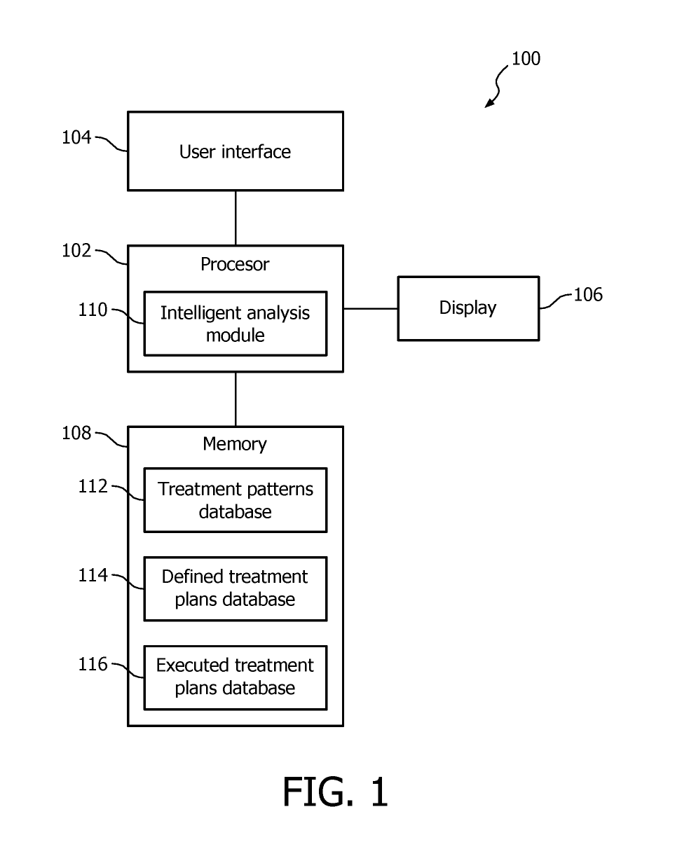

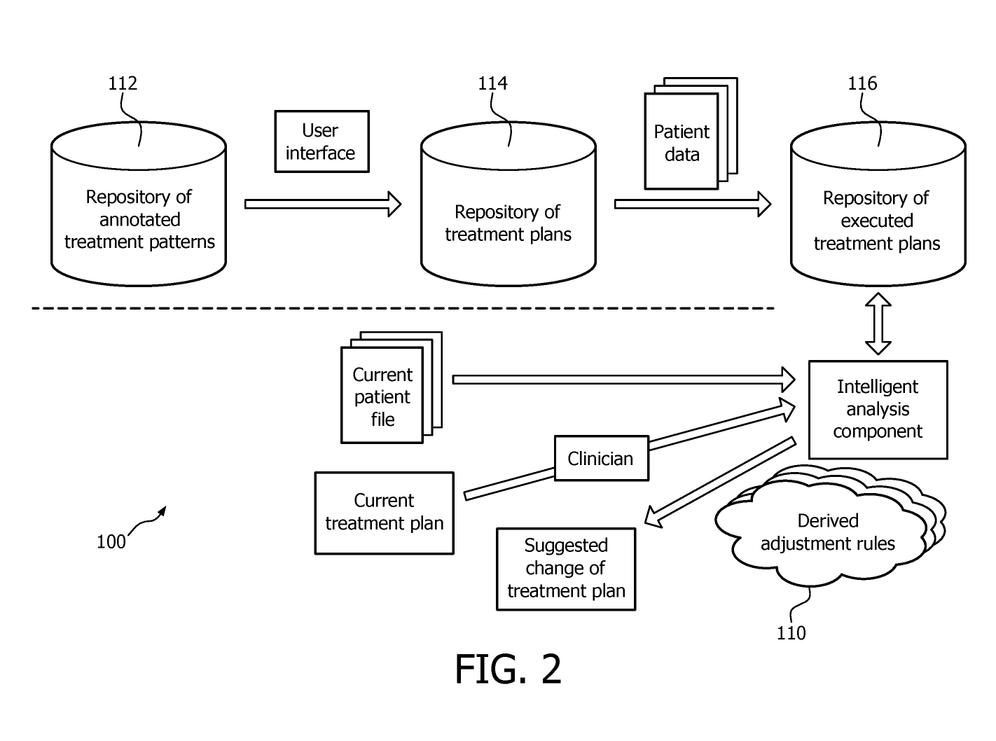

As shown in FIGS. 1 and 2, a system 100 according to an exemplary embodiment of the present disclosure generates recommendations for a treatment plan for a patient based on prior treatment plans and specific patient data. The provided recommendations are used to generate and manage a current treatment plan for a particular patient. The system 100 comprises a processor 102, a user interface 104, a display 106 and a memory 108. The processor 102 includes an intelligent analysis module 110 which analyzes annotated treatment patterns stored in a treatment patterns database 112, defined treatment plans stored in a defined treatment plans database 114 and/or executed treatment plans stored in an executed treatment plans database 116 along with specific patient data (e.g., conditions, tests and results) to generate recommendations for a user regarding a current treatment plan for a particular patient. The current treatment plan is generated based on the provided recommendations and user input, which may be inputted via the user interface 104. The user interface 104 may include input devices such as, for example, a keyboard, a mouse and/or touch display on the display 106. The recommendations provided by the intelligent analysis module and/or the generated current treatment plan may be displayed on the display 106.

The treatment patterns database 112, defined treatment plans database 114 and executed treatment plans database 116 are stored in the memory 108 and include patterns/plans for a specific department or disease domain such that recommendations to the user closely fit the context of the desired current treatment plan. For example, the system 100 may be configured to provide recommendations in regard to an oncology outpatient treatment. It will be understood by those of skill in the art, however, that the system 100 may be configured for any of a variety of departments or diseases. The treatment patterns database 112 may include annotated treatment patterns extracted from, for example, available free text treatment plans. The defined treatment plans database 114 includes treatment plans that have been previously generated based on the recommendations provided by the intelligent analysis module 110. The executed treatment plans database 116 includes defined treatment plans that have been updated, edited and/or annotated to reflect changes to the treatment plan during execution. The intelligent analysis module 110 analyzes the patterns and plans from the treatment patterns database 112, the defined treatment plans database 114 and/or the executed treatment plans database 116 based on specific patient data to generate recommendations regarding the current treatment plan for the patient. Those skilled in the art will understand that it is not necessary to store the described data in three separate databases 112, 114, 116 as this construct is only used to logically separate the different types of data.

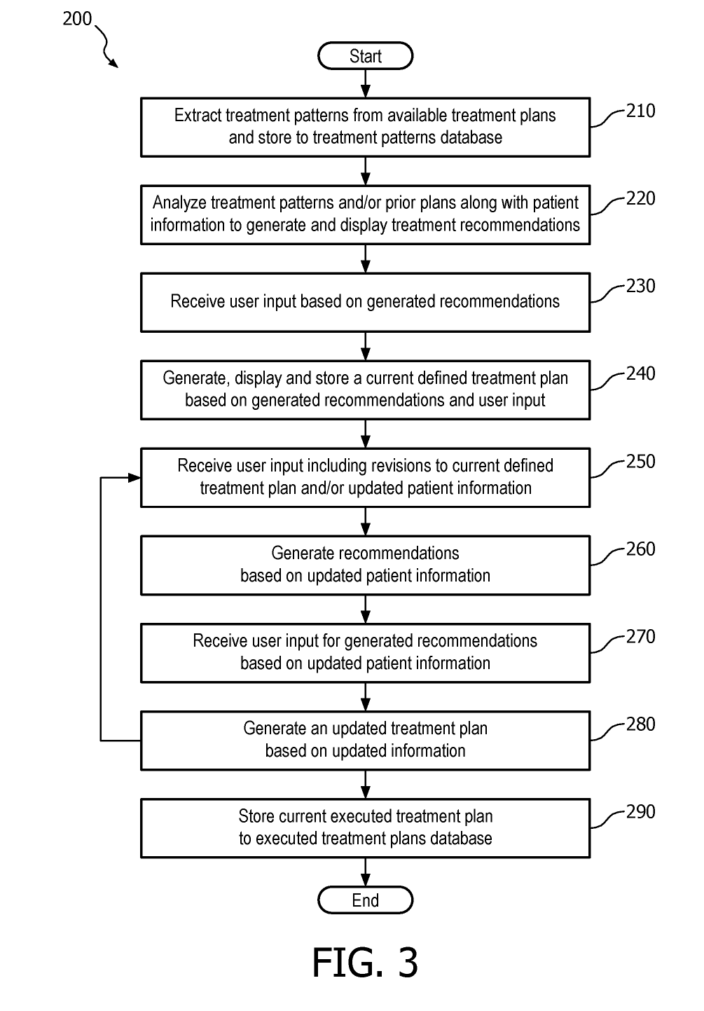

FIG. 3 shows a method 200 for generating recommendations for a current treatment plan for a patient having a particular condition or disease and managing the current treatment plan based on the evolving needs of the patient, using the system 100 described above. The exemplary embodiment describes generating treatment plan recommendations for a patient within an oncology outpatient program. In a step 210, the processor 102 extracts treatment patterns from available free text treatment plans regarding a particular department or disease and stores the extracted treatment patterns in the treatment patterns database 112. The available treatment plans may include, for example, prior treatment plans stored in the defined treatment plans database 114 and/or the executed treatment plans database 116 along with treatment plans available to and accepted by clinicians from a variety of sources. These treatment patterns are extracted by comparing the available treatment plans and identifying recurring patterns. The treatment patterns may also be annotated to include specific drugs and dosages as well as reasons for a particular course of treatment so that recommendations may be made based on these reasons and specific patient information.

Treatment patterns may be identified, for example, by knowledge experts who review a representative number and range of treatment plans and suggest relevant patterns. The processor 102 may then evaluate the coverage of the suggested patterns in the entire set of available treatment plans using information extraction techniques such as, for example, a Natural Language Processing (NLP) module and regular expressions found in the patterns. The evaluation will show whether the identified treatment patterns can be detected within the available treatment plans. The treatment patterns may be stored in the treatment patterns database 112 and/or new patterns may be defined as a result of the evaluation. For example, in all treatment plans including an antibiotic, a pattern may be identified and annotated with the particular substance/drug and the dosage. Patterns may then be reused across plans and modified/annotated, as necessary. An annotated pattern for a plan including an antibiotic may be, for example, Doxycycline with a dosage of 100mg every 12 hours for 10 days, which may be reused across plans. The pattern, however, may be modified, when necessary, to instead include Tetracycline every 6 hours for 5 days, depending on the circumstance and specific patient information.

The intelligent analysis module 110 analyzes treatment patterns stored in the treatment patterns database 112 and/or prior defined treatment plans stored in the defined treatment plans database 114 and/or prior executed treatment plans stored in the executed treatment plans database 116 along with patient information (e.g., conditions, tests and results) to generate recommendations regarding the current treatment plan, in a step 220, and display the recommendations on the display 106. For example, if the clinician requires an antibiotic pattern for treating pneumonia, the processor 102 may retrieve instantiated patterns previously used for the same disease and recommend Doxycycline 2ith a dosage of 100mb every 12 hours for 10 days. In initial runs through the method 200 in which the defined and executed treatment plans databases 114, 116 have not yet been populated, it will be understood by those of skill in the art that the intelligent analysis module 110 may only analyze treatment patterns and patient information to generate recommendations for a current treatment plan. In subsequent runs through the method 200, however, it will be understood by those of skill in the art that the intelligent analysis module 110 may also analyze prior defined and executed treatment plans stored in the defined treatment plans database 114 and the executed treatment plans database 116, respectively, to generate recommendations for current treatment plan. As will be described in further detail below, the defined treatment plans database 114 and the executed treatment plans database 116 will be continuously updated with more current treatment plan information for generating treatment plan recommendations. Treatment recommendations may be displayed on the display 106 for viewing by a user (e.g., clinician). The recommendations may be displayed in, for example, a pre-defined form so that the user may easily select desired recommendations. It will be understood by those of skill in the art that the recommendations may be displayed in any of a variety of formats so long as the user may easily determine and input which of the recommendations are desired for the current treatment plan.

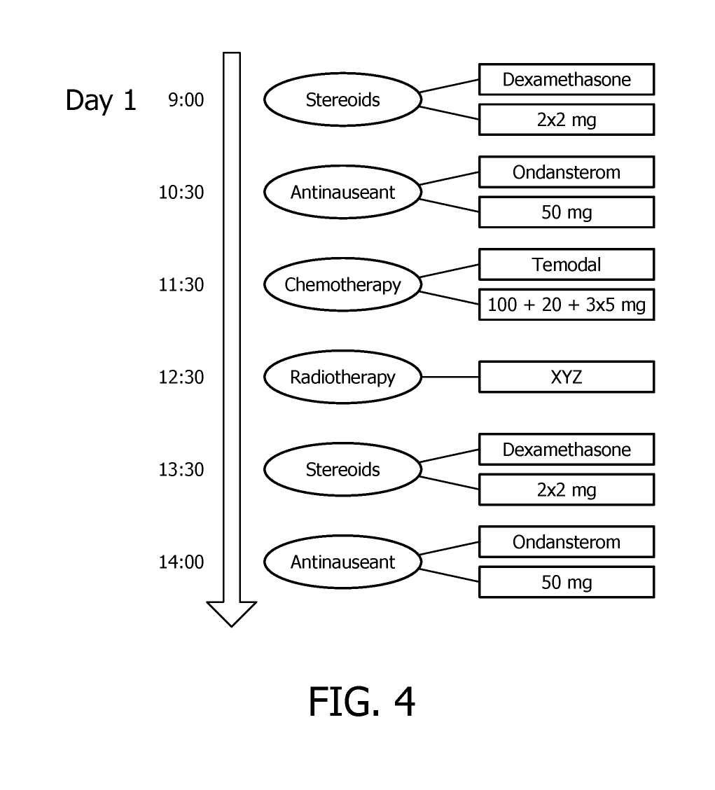

The user may select the desired recommendations and/or provide additional user input regarding the current treatment plan, in a step 230, using the user interface 104. The user input may include, for example, selecting which of the recommendations to include within the current treatment plan, a timeline for the selected steps of the current treatment plan and specific medications and/or dosages for steps of the treatment plan which require them. Based on the user input, the processor 102 generates a current defined treatment plan, in a step 240. The current defined treatment plan may be stored in the defined treatment plans database 114 and displayed on the display 106 for viewing by the user. In an exemplary embodiment the specific medications and dosages may included as annotations to the steps within the treatment plan. An exemplary defined treatment plan is shown in FIG. 4. The current defined treatment plan may include, for example, a timeline outlining the various steps of the treatment plan and the specific medications and/or dosages required for the steps.

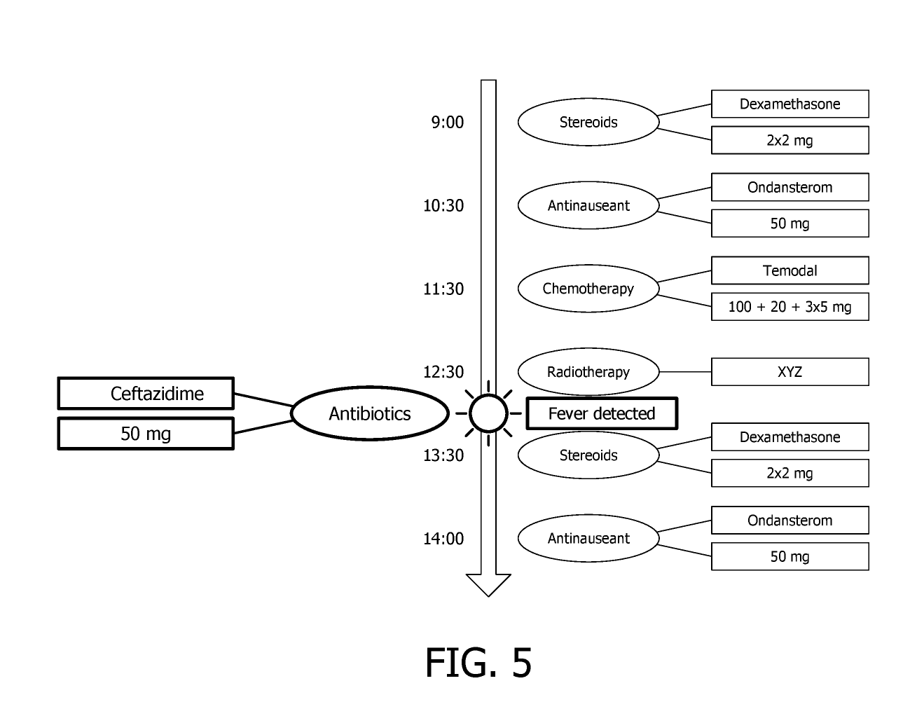

In a step 250, the user may input revisions to the current defined treatment plan and/or input new/updated information which may necessitate a change in the current defined treatment plan. For example, the patient may experience a new event such as a fever or results of a test may be inputted to determine whether a change is required. Where the user has inputted information that requires a change to the current defined plan, the intelligent analysis module provides a recommendation to the user, in a step 260, based on the new information and rules which have been established and stored in the treatment patterns database 112. For example, the intelligent analysis module 110 may determine that treatment patterns have shown that in instances where patients have developed a fever during the course of treatment, an antibiotic was administered. The generated recommendation may also indicate specific antibiotics and/or dosages that have been administered for particular situations so that the user may select a specific course of treatment, in a step 270. Based on the user input provided in the step 270, the processor 102 generates an updated treatment plan which may be displayed on the display 106, in a step 280. The updated treatment plan may also be annotated to include reasons for the change to the current defined treatment plan. For example, the updated treatment plan may include an annotation that shows that the treatment plan was updated to include administration of an antibiotic to treat a fever. An exemplary updated treatment plan is shown in FIG. 5. It will be understood by those of skill in the art, steps 250-280 may be repeated, as necessary during the patient's course of treatment to revise, edit and update the current treatment plan with new and/or additional information. It will also be understood by those of skill in the art that where the user enters a revision which does not require a recommendation, in the step 250, the method 200 may proceed directly to the step 280 in which an updated treatment plan reflecting the revision is displayed on the display 106.

Upon completion of the course of treatment defined for the patient, a current executed treatment plan including all the additional information, updates and changes to the current defined treatment plan reflected in all iterations of the updated treatment plan, is stored to the executed treatment plans database 116. Once the current executed treatment plan is stored to the executed treatment plans database 116, it will be understood by those of skill in the art that the current executed treatment plan becomes prior information which may be considered during the extraction of patterns in the step 210 and the analysis of patterns and/or plans in the step 220 when the method 200 is repeated for another patient within the same department/disease domain.

It is noted that the claims may include reference signs/numerals in accordance with PCT Rule 6.2(b). However, the present claims should not be considered to be limited to the exemplary embodiments corresponding to the reference signs/numerals.

Those skilled in the art will understand that the above-described exemplary embodiments may be implemented in any number of manners, including, as a separate software module, as a combination of hardware and software, etc. For example, the intelligent analysis module 110 may be a program containing lines of code that, when compiled, may be executed on a processor.

It will be apparent to those skilled in the art that various modifications may be made to the disclosed exemplary embodiments and method and alternatives without departing from the spirit or scope of the disclosure. Thus, it is intended that the present disclosure cover the modifications and variations provided that they come within the scope of the appended claims and their equivalents.

1. A method for managing an executable medical treatment plan, comprising:
analyzing at least one of treatment patterns corresponding to recurring patterns in treatment plans and prior treatment plans in view of patient information to generate treatment plan recommendations using a processor;
receiving a user input regarding the treatment plan recommendations via a user interface; and
generating a current defined treatment plan based on the treatment plan recommendations and the user input regarding the treatment plan recommendations using the processor.

2. The method of claim 1, further comprising:
storing the current defined treatment plan to a defined treatment plan database.

3. The method of claim 1, further comprising:
displaying the current defined treatment plan on a display.

4. The method of claim 1, further comprising:
receiving second user input including one of revisions to the current defined treatment plan and updated patient information.

5. The method of claim 4, further comprising:
analyzing the updated patient information and treatment patterns to generate updated treatment recommendations using the processor.

6. The method of claim 5, further comprising:
receiving third user input regarding the updated treatment recommendations and generating an updated treatment plan based on the third user input regarding the updated treatment recommendations.

7. The method of claim 6, further comprising:
storing a current executed treatment plan including updates of the updated treatment plan in an executed treatment plans database.

8. The method of claim 7, wherein the current executed treatment plan includes annotations describing reasons for changes in the current defined treatment plan.

9. The method of claim 1, wherein treatment patterns are extracted from available treatment plans including prior treatment plans.

10. A system for managing an executable medical treatment plan, comprising:
a memory storing treatment patterns in a treatment patterns database and a set of instructions; and
a processor executing the instructions which causes the processor to perform operations comprising:
analyzing at least one of treatment patterns corresponding to recurring patterns in treatment plans and being stored in the treatment patterns database and prior treatment plans in view of patient information to generate treatment plan recommendations using a processor;
receiving a user input regarding the treatment plan recommendations; and
generating a current defined treatment plan based on the treatment plan recommendations and the user input regarding the treatment plan recommendations using the processor.

11. The system of claim 10, wherein the memory includes a defined treatment plans database storing prior defined treatment plans and the current defined treatment.

12. The system of claim 10, further comprising:
a display displaying the current defined treatment plan.

13. (canceled)

14. (canceled)

15. (canceled)

16. The system of claim 10, wherein the memory includes an executed treatment plan database storing prior executed treatment plans and the current executed treatment plan, which includes updates of the updated treatment.

17. (canceled)

18. (canceled)

19. The system of claim 10, wherein the treatment patterns include annotations describing situations in which a particular treatment pattern is relevant.

20. A non-transitory computer-readable storage medium including a set of instructions executable by a processor, the set of instructions, when executed by the processor, causing the processor to perform operations comprising:
analyzing at least one of treatment patterns corresponding to recurring patterns in treatment plans and prior treatment plans in view of patient information to generate treatment plan recommendations using a processor;
receiving a user input regarding the treatment plan recommendations via a user interface; and
generating a current defined treatment plan based on the treatment plan recommendations and the user input regarding the treatment plan recommendations using the processor.
